(19) United States
(12) Patent Application Publication
(10) Pub. No.: US 2026/0108523 A1
(43) Pub. Date: Apr. 23, 2026

(54) FORMULATION AND A METHOD OF TREATING CARCINOMA

(71) Applicants: Sravathi AI Technology Pvt Ltd, Bangalore (IN); MAYO FOUNDATION FOR MEDICAL EDUCATION AND RESEARCH, Rochester, MN (US)

(72) Inventors: Nagaraj Kulkarni, Bangalore (IN); Bhargavi Baskaran, Bangalore (IN); Ben Geoffrey Arul Samraj, Bangalore (IN); Raghu Bhagavat, Bangalore (IN); Kishan Gurram, Bangalore (IN); Debabrata Mukhopadhyay, Jacksonville, FL (US); Hari Krishnareddy Rachamala, Jacksonville, FL (US); Naga M. Nakka, Jacksonville, FL (US); Krishnendu Pal, Jacksonville, FL (US)

(21) Appl. No.: 19/366,141

(22) Filed: Oct. 22, 2025

Related U.S. Application Data
(60) Provisional application No. 63/711,062, filed on Oct. 23, 2024.

(51) Int. Cl. A61K 31/517; A61K 31/17; A61K 31/426; A61K 31/427; A61K 31/513; A61K 31/519; A61K 31/52; A61K 31/551; A61K 31/706; A61K 31/7068; A61K 31/7072; A61K 31/7076; A61P 35/00

(52) U.S. Cl. CPC A61K 31/517; A61K 31/17; A61K 31/426; A61K 31/427; A61K 31/513; A61K 31/519; A61K 31/52; A61K 31/551; A61K 31/706; A61K 31/7068; A61K 31/7072; A61K 31/7076; A61P 35/00

(57) ABSTRACT

A formulation and a method of treatment for mitigating pancreatic and renal carcinoma are disclosed. The method includes administering in a subject in need thereof, a formulation containing a partial agonist of PPAR-gamma, for example, balaglitazone. Also, balaglitazone exhibits synergistic anticancer activity when used in combination with a chemotherapy drug.

### CROSS-REFERENCE TO RELATED APPLICATION

The instant application claims priority to U.S. Provisional Patent Application Ser. No. 63/711,062, filed Oct. 23, 2024, the entire specification of which is expressly incorporated herein by reference.

### FIELD OF THE INVENTION

The present invention relates generally to cancer treatment therapies, and more particularly to formulations and methods of treating a carcinoma comprising the step of administering to a subject a formulation, wherein the formulation comprises a formulated first active compound, wherein the first active compound is a thiazolidinedione-based partial agonist of PPAR-γ, and optionally, a synergistic combination of the formulated first active compound and a formulated second active compound, wherein the second active compound is a chemotherapy drug.

### BACKGROUND OF THE INVENTION

Peroxisome proliferator-activated receptors (PPAR) are nuclear hormone receptors which are categorized into three subtypes, α, δ(β), and γ. Among them, PPAR-γ has been long known to be responsible for major functions such as controlling metabolism, cellular differentiation and cellular development in humans. It is expressed across a range of tissues, including adipose tissue, vascular cells, fibroblasts, and immune cells. PPAR-γ regulates body metabolism by responding to intake of dietary lipids [see REF. 1]. It plays an important role in diabetes by fat modulation and storage and metabolism of carbohydrates [see REF. 2]. The importance of beneficial role of PPAR-γ underscores the role for agonists of these receptors.

Principal natural ligands/agonists of PPAR-γ include dietary supplements like lipids and their metabolic derivatives. Since the 1980s, several types of ligands which act as synthetic PPAR-γ agonists have been found for the treatment of many diseases, particularly diabetes. Well known synthetic PPAR-γ agonists belong to thiazolidinedione (TZD) class of compounds, examples of which include first generation TZDs, such as ciglitazone and troglitazone, followed by second generation TZDs, such as pioglitazone (PIO) and rosiglitazone (RGZ). Some of these have been shown to be reasonably successful in the treatment of diabetes [see REF. 3]. Both monotherapy as well as combinations with other types of antidiabetic medication are known for these thiazolidinediones.

Ciglitazone was withdrawn from the market due to its liver toxicity. Troglitazone was the first approved glitazone for decreasing glycemia in type 2 diabetes patients; however, it was removed from the market due to its hepatotoxicity issues and liver damage risk [see REFS. 4-5]. Pioglitazone, also approved to treat type 2 diabetes, has been withdrawn from the market in Germany and France due to the risk of bladder cancer; in other countries it has been advised to include warning about bladder cancer in labelling the drugs containing pioglitazone [see REF. 6]. Rosiglitazone is an insulin sensitizing agent; it was approved for glycemic control with type 2 diabetes and was there in the market for some time before being withdrawn from the market due to its risk of causing ischemic heart disease [see REFS. 7-8]. The reports on sporadic side effects particularly, of using those thiazolidinediones which are known as full agonists of PPAR-γ act as a deterrent in the widespread use of these compounds in the treatment of diabetes.

Partial agonists of PPAR-γ are also known and have also been found active in the treatment of diabetes, though none of these have been approved. Among the known partial agonists, examples of thiazolidinediones based agonists are netoglitazone, GQ-16 ((5Z)-5-(5-bromo-2-methoxy-benzylidene)-3-(4-methyl-benzyl)-thiazolidine-2,4-dione), balaglitazone and efatutazone. In the case of balaglitazone, it is well established that side effects are much lower than those for the other common thiazolidinediones studied [see REFS. 9-10]. Balaglitazone was previously tested clinically for treating type 2 diabetes. It was not moved forward after clinical phase-3, because it was not better than other existing PPAR-γ agonists in terms of efficacy against diabetes. Balaglitazone is considered as a weak activator when compared with the full agonists and exhibits less transactivation potential and yet delivers desirable effects [see REF. 11]. In clinical studies for treating type 2 diabetes, balaglitazone was tested with doses of 10 mg and 20 mg/day and reported to show hardly any adverse side effects [see REFS. 12-14].

The role of PPAR-γ receptor and its full agonists in cancer

Based on recent studies, PPAR-γ receptor has a prominent role in cancer mitigation as well. PPAR-γ is present in different types of tumor cells, and activation of PPAR-γ receptor leads to decrease in proliferation of cell or causes cell death [see REFS. 15-16]. Many mechanisms have been proposed for role of PPAR-γ in cancer, including a) upregulation of cancer suppressor genes and causing cell cycle arrest and b) activation of major pathways of inflammatory and immune responses. A few of the thiazolidinediones (TZD), known as full agonists of PPAR-γ, have been shown to be active against different types of cancer possibly due to appropriate activation of PPAR-γ; related reports are summarized herein.

Ciglitazone has been tested to inhibit cyclin D1 through in vitro tests requiring high concentration of ciglitazone i.e., up to 60 μM to induce activity [see REF. 17]. Besstina E. Lyles et al., have shown glitazones (ciglitazone) to have anti-cancer effects both in PPAR-γ dependent and independent manner [see REF. 18]. Thomas Botton et al., conducted experiments both in vitro and in vivo and found that ciglitazone inhibits human melanoma xenograft [see REF. 19]. Youngsoo kim et al., reported that ciglitazone showed activity against prostate cancer cells but for a lack of specificity in recognizing Bcl-xL and Bcl-2 [see REF. 20]. In another study, Chung-Wai Shiau et al., observed that with ciglitazone, NF-Kb levels (important for immune response) were not reduced in tumor cells [see REF. 21]. Chunhua Qin et al., observed that ciglitazone acts as anti-cancer compound in breast cancer only at high concentrations see REF. [22]. E Pestereva et al., observed that ciglitazone showed good activity in the in-brain tumor stem cells and reduced glioma [see REF. 23].

Troglitazone has been reported to inhibit cellular viability in PC-3 prostate cancer cells through in vitro studies on different cell lines [see REF. 24]. In follicular carcinoma and breast cancer cell lines, troglitazone tested in presence of retinol and retinoic acid, revealed the dual role of differentiation and inhibition of proliferation [see REFS. 25-26]. In human pancreatic cancer cell lines, troglitazone activity was moderate in decreasing cell migration [see REF. 27]. In NSCLC, chemo resistant ovarian, gastric carcinoma and cervical cancer cell lines, troglitazone tested in combinations with cisplatin, heregulin and RXR agonist, showed significant synergistic effect [see REFS. 28-32]. Elisabetta Mueller et al., has conducted phase-II clinical trial and confirmed that no symptoms of metastasis was observed in prostate cancer [see REF. 33]. A study done by Hiroyuki Kohno et al., for troglitazone on chemically-induced Aberrant Crypt Foci in Rats has shown results of decrease of neoplastic lesions and mitigation of colon tumorigenesis [see REF. 34]. Studies by Koujiro Yoshida et al on rat tongue carcinogenesis, initiated with 4-nitroquinoline 1-oxide showed that dietary troglitazone decreased oral carcinogenesis [see REF. 35].

Studies of Pioglitazone on gliomas, has revealed that concentration of the pioglitazone used is critical; at low concentrations it does not show good efficacy or inhibitory effect on cell viability [see REF. 36]. Studies reported by Itasu Ninomiya et al., on pancreatic cell lines showed that, at >10 μM of pioglitazone showed significant effect on reducing cell proliferation and prevented metastasis [see REF. 37]. Yuan Yang et al., have shown effects of pioglitazone on Hepatocellular carcinoma cell lines; in most of the cell lines, the activity was the same at different concentrations and in some cell lines pioglitazone exhibited activity only at higher concentrations [see REF. 38]. Nishi Srivastava et al., have identified anti-tumorigenic effects of PPAR-γ agonists by using pioglitazone [see REFS. 39-40]. There are also two clinical trials involving pioglitazone during and after radiation therapy in patients with brain tumors; pioglitazone and rofecoxib were combined with angiostatically scheduled trofosfamide in the treatment of far-advanced melanoma and soft tissue sarcoma resulting in the observation that pioglitazone prevented radiation-induced cognitive decline and stabilized remission respectively [see REFS. 41-43].

Studies reported on rosiglitazone on liver cancer concluded that rosiglitazone sensitizes 5-flouro uracil antitumor activity in HCC cell lines; When 5-FU is co-administered with 10, 30, and 50 μmol/L rosiglitazone to cell lines for 48 h, rosiglitazone showed increase in antitumor activities. In Jabl overexpressed liver cancer, rosiglitazone administration inhibits cell proliferation. Rosiglitazone induces apoptosis of cells through TRAIL [see REFS. 44-46]. In human melanoma cell line A375, rosiglitazone promotes apoptosis and decreased colony formation and there is evidence for G1 phase of cells [see REF. 47]. Alan Prem Kumar et al., and Miguel Pignatelli et al., have reported studies of rosiglitazone on breast cancer and identified that combination of rosiglitazone with chemotherapeutic agents has therapeutic effects for breast cancer [see REFS. 48-49]. In pancreatic cancer, rosiglitazone in combination with cyclooxygenase-2 inhibitor or gemcitabine showed enhanced cell apoptosis and inhibition of cell proliferation [see REFS. 50-51]. In Non-Small Cell Lung Cancer (NSCLC), rosiglitazone has shown activity by regulating 15-hydroxyprostaglandin dehydrogenase and through activation of Tumor Sclerosis Complex-2 [see REFS. 52-53]. Rosiglitazone causes cell cycle deregulation in adrenocortical cancer cells and also initiated G0/G1 phase arrest of cell cycle [see REF. 54]. Liang Qin et al., identified that rosiglitazone causes inhibition of the prostate cancer cell lines by inhibiting CXCR4/CXCL12 axis [see REF. 55]. In human colorectal cancer, Ruizheng Miao et al., reported that combination of rosiglitazone with retinoic acid inhibits the COX-2, MMP-7 and TIMP-1 expression, there by inhibiting cell proliferation [see REF. 56]. Aaron G. Smith et al., reported rosiglitazone reduces cell proliferation through the WNT signaling. Mingzhong Rui et al., reported rosiglitazone inhibits angiogenesis in myeloma cell lines by downregulating hypoxia-inducible factor-la and insulin-like growth factor-1 mRNA expression [see REFS. 57-58].

From the above summary of reports on TZD molecules for cancer treatment in in vitro and in vivo studies, it is apparent that only in high concentrations, they work in inhibition of cell proliferation and in showing anti-tumorigenic effects. However, higher concentrations lead to undesirable side effects. Most of the available anticancer reports on TZD molecules are based on in vitro studies and reports on in vivo studies are comparatively less in treatment of cancer.

As already mentioned, PPAR-γ regulates metabolism of lipid and glucose, inhibits inflammation and immunity responses, suppresses cell proliferation and induces cell differentiation and apoptosis. However, evidence is also available to indicate that PPAR-γ appears to play contradictory role in the control of cancer [see REF. 59]. While PPAR-γ appears to show great potential in the inhibition of tumor proliferation and metastasis, there are examples wherein, PPAR-γ appears to promote tumorigenesis by enhancing intercellular adhesion and by restraining apoptosis. In several studies, with the cell culture of human renal cell carcinoma, human prostate cancer xenografts in nude mice, as well as brain cancer in vivo, the increased expression of PPAR-γ are all prognostically negative and pro-metastatic. Hence the role of PPAR-γ agonists in cancer is to be well calibrated and understood.

Role of “TZD” Partial Agonists of PPAR-γ in cancer

TZD stands for thiazolidinedione fragment. Partial agonists of PPAR-γ can be based on TZD or non-TZD fragments. Many full agonists containing TZD fragments appear to be reduce cancer cell proliferation in different cancers. However, side effects and toxicity exhibited by the full agonists prevent them from becoming the panacea desired. Many of the TZD compounds have been taken away from the market owing to undesired side effects. In this context, the role of partial agonists containing thiazolidinedione in providing a plausible treatment for various cancers is yet to be fully studied and understood.

One of the partial agonists containing thiazolidinedione fragment is balaglitazone. In the studies on treatment of diabetes, balaglitazone did not show activity advantage over other the full agonists of PPAR-γ, despite exhibiting little undesirable side effects in the studies. Possibly stemming from a perception due to reduced activity in diabetes treatment studies, studies on the role of balaglitazone in anticancer studies is limited.

Studies reported by Bahman Yousefi et al., reveal that when balaglitazone is tested in doxorubicin resistant human myelogenous leukemia, it reduces the resistance to the doxorubicin in doxorubicin resistant cancers in chemotherapy of cancerous patients [see REF. 60].

In a patent publication, WO2018132899A1, many combinations of balaglitazone with a broad list of inflammatory agents and chemotherapeutic agents like cisplatin for preventing osteosarcoma lung metastasis have been suggested [see REF. 61].

Another molecule of TZD family, netoglitazone (also called MC-555), an experimental drug molecule, is known to be a full agonist, partial agonist or antagonist of PPAR-γ in a cell type specific manner. A Phase-2 clinical trial on netoglitazone related to type 2 diabetes was discontinued following an instance of carcinogenicity [see REF. 62]. However, there is a report on netoglitazone exhibiting anti-proliferative activity in pancreatic cancer cells, both by PPAR-γ dependent and independent manner [see REF. 63]. It is also reported that MCC-555 demonstrated an apoptotic activity in human colorectal cancer cells [see REF. 64].

GQ-16 has been studied against cancer. GQ-16 has proved to be more potent than the full agonist rosiglitazone on reducing the viability of breast cancer cells line MCF-7 [see REF. 65]. Selective modulation of PPAR-γ by GQ-16 is effective in reducing proteinuria as well as comorbidities associated with nephrotic syndrome, while also providing reduced potential for adipogenesis [see REF. 66].

Yet another PPAR-γ partial oral agonist efatutazone has been reported to be active against thyroid cancer with nanomolar activity when used in combination with paclitaxel [see REF. 67]. Efatutazone's clinical trials for colorectal cancer have been discontinued, for reasons of low efficacy [see REF. 68].

‘Non-TZD’ partial agonists of PPAR-γ [see REF. 69] in cancer

Some of the other known experimental or investigational partial agonists of PPAR-γ which are not based on thiazolidinedione (TZD) are mentioned below:
PAR-1622: (S)-2-ethoxy-3 (4-(5-(4-(5-(methoxymethyl) isoxazol-3-yl)phenyl)-3-methylthiophen-2-yl) methoxy)phenyl) propanoic acid;
PAM-1616: (S)-2-ethoxy-3-(4-((3-methyl-5-(4-(3-methylisoxazol-5-yl)phenyl) thiophen-2-yl) methoxy)phenyl) propanoic acid;
FK614: 3-(2,4-dichlorobenzyl)-2-methyl-N-(pentylsulfonyl)-3-H-benzimidazole-5-carboxamide;
F12016: 2-[2-(1,2-dimethyl-1H-indol-3-yl)-2-oxo-acetylamino]-benzamide; and telmisartan.

Among these, telmisartan is an approved drug and considered as a partial agonist of PPAR-γ and it has been found to show anticancer effects in diverse types of cancer cells [see REFS. 70-71].

It is common knowledge that treatments developed for one type of cancer may not work for another type of cancer. Despite many years of research, and a multitude of treatments available to cancer patients, there is still a long felt need in the art for additional cancer therapies. Among the various cancer diseases, pancreatic cancer is among the hard ones to treat and manage. It is ranked 14th in incidence and 7th in mortality in the world [see REF. 72]. In USA, about 66,440 people (34,530 men and 31,910 women) are expected to be diagnosed with pancreatic cancer. About 51,750 people (27,270 men and 24,480 women) may die of pancreatic cancer according to reports of American cancer society in 2024 [see REF. 73]. In India, the incidence of pancreatic cancer is 0.5-2.4/100,000 persons per year in women and 0.2-1.8/100,000 persons per year in men [see REF. 74].

According to Oxford academy reports, kidney cancer (KC) is a disease with a rising worldwide incidence estimated at 400 000 new cases annually, and a worldwide mortality rate approaching 175 000 deaths per year [see REFS. 75-77]. Humanity is in dire need of an effective therapy for treating these maladies.

Accordingly, there exists a need for novel therapies for the treatment of carcinomas, particularly pancreatic and kidney cancers, which overcome the aforementioned deficiencies.

### SUMMARY OF THE INVENTION

The following presents a simplified summary to provide a basic understanding of the present invention. This summary is not an extensive overview of the present invention and is not intended to identify key features of the invention. Rather, it is to present some of the inventive concepts of this invention in a generalised form as a prelude to the detailed description and claims that are to follow.

The present invention pertains to a formulation and a method of treating a carcinoma selected from a group consisting of renal carcinoma and pancreatic carcinoma in a subject in need thereof, the method comprising administering to the subject a composition including a first active compound wherein, the first active compound is a thiazolidinedione-based partial agonist of PPAR-γ or optionally, a synergistic combination of the first active compound and a second active compound wherein, the second active compound is a chemotherapy drug.

The first active compound is a molecule selected from a group consisting of balaglitazone, netoglitazone and a combination thereof and the second active compound is a molecule selected from a group consisting of gemcitabine, 5-fluorouracil, 6-mercaptopurine, azacitidine, capecitabine, cladribine, clofarabine, cytarabine, decitabine, floxuridine, fludarabine, hydroxyurea, methotrexate, nelarabine, pemetrexed, pentostatin, pralatrexate, thioguanine, trifluridine/tipiracil combination and a combination thereof.

In accordance with a first embodiment of the present invention, there is provided a method of treating a carcinoma in a subject in need thereof, wherein the carcinoma includes renal carcinoma or pancreatic carcinoma, comprising the step of:
administering to the subject a formulation;
wherein the formulation comprises:
   a formulated first active compound;
   wherein the first active compound is a thiazolidinedione-based partial agonist of PPAR-γ; and
   optionally, a synergistic combination of the formulated first active compound and a formulated second active compound;
   wherein the second active compound is a chemotherapy drug.

In accordance with an aspect of the first embodiment, the formulation is a solid or a liquid in form.

In accordance with an aspect of the first embodiment, the formulated first active compound is administered orally or parenterally.

In accordance with an aspect of the first embodiment, the formulated second active compound is administered parenterally.

In accordance with an aspect of the first embodiment, the parenteral administration comprises a subcutaneous injection, an intraperitoneal injection, an intravenous injection, an intravenous infusion or an intramuscular injection.

In accordance with an aspect of the first embodiment, the formulation is a synergistic combination of the formulated first active compound and the formulated second active compound, the formulated first active compound and the formulated second active compound of the composition are administered simultaneously, are administered sequentially, are administered in alternating series or are administered in temporal proximity.

In accordance with an aspect of the first embodiment, the first active compound is a molecule selected from the group consisting of balaglitazone, efatutazone, GQ-16, netoglitazone, and combinations thereof.

In accordance with an aspect of the first embodiment, the second active compound is a molecule selected from the group consisting of gemcitabine, 5-fluorouracil, 6-mercaptopurine, azacitidine, capecitabine, cladribine, clofarabine, cytarabine, decitabine, floxuridine, fludarabine, hydroxyurea, methotrexate, nelarabine, pemetrexed, pentostatin, pralatrexate, thioguanine, trifluridine, tipiracil, and combinations thereof.

In accordance with an aspect of the first embodiment, the formulated first active compound or formulated second active compound further comprises one or more pharmaceutically acceptable carriers, excipients, or diluents.

In accordance with an aspect of the first embodiment, the subject is a mammal.

In accordance with an aspect of the first embodiment, the mammal is selected from the group consisting of mice, monkeys, rats, rabbits, and combinations thereof.

In accordance with a second embodiment of the present invention, there is provided a formulation for treating a carcinoma in a subject in need thereof, wherein the carcinoma includes renal carcinoma or pancreatic carcinoma, comprising:
a formulated first active compound;
wherein the first active compound is a thiazolidinedione-based partial agonist of PPAR-γ; and
optionally, a synergistic combination of the formulated first active compound and a formulated second active compound;
wherein the second active compound is a chemotherapy drug.

In accordance with an aspect of the second embodiment, the formulation is a solid or a liquid in form.

In accordance with an aspect of the second embodiment, the formulated first active compound is administered orally or parenterally.

In accordance with an aspect of the second embodiment, the formulated second active compound is administered parenterally.

In accordance with an aspect of the second embodiment, the parenteral administration comprises a subcutaneous injection, an intraperitoneal injection, an intravenous injection, an intravenous infusion or an intramuscular injection.

In accordance with an aspect of the second embodiment, when the formulation is a synergistic combination of the formulated first active compound and the formulated second active compound, the formulated first active compound and the formulated second active compound of the composition are administered simultaneously, are administered sequentially, are administered in alternating series or are administered in temporal proximity.

In accordance with an aspect of the second embodiment, the first active compound is a molecule selected from the group consisting of balaglitazone, efatutazone, GQ-16, netoglitazone, and combinations thereof.

In accordance with an aspect of the second embodiment, the second active compound is a molecule selected from the group consisting of gemcitabine, 5-fluorouracil, 6-mercaptopurine, azacitidine, capecitabine, cladribine, clofarabine, cytarabine, decitabine, floxuridine, fludarabine, hydroxyurea, methotrexate, nelarabine, pemetrexed, pentostatin, pralatrexate, thioguanine, trifluridine, tipiracil, and combinations thereof.

In accordance with an aspect of the second embodiment, the formulated first active compound or formulated second active compound further comprises one or more pharmaceutically acceptable carriers, excipients, or diluents.

In accordance with an aspect of the second embodiment, the subject is a mammal.

In accordance with an aspect of the second embodiment, the mammal is selected from the group consisting of mice, monkeys, rats, rabbits, and combinations thereof.

Specific variations of the method for administering the first active compound or a combination of first and second active compound are also disclosed. The surprising effectiveness of the method in mitigating pancreatic cancer or kidney cancer in a subject is also disclosed.

### BRIEF DESCRIPTION OF THE DRAWINGS

This invention will be described by way of non-limiting embodiments of the present invention, with reference to the accompanying drawings, in which:

FIGS. 1A to 1D schematically illustrate the inoculation schedule and treatment regime and anti-tumor effects of SAIT-DRP-001 in 786-O tumor-bearing renal xenograft mouse models. Mice bearing 786-O tumors were treated with two different treatment groups, control and SAIT-DRP-001. The word ‘Figure’ is equivalently presented as ‘Fig.’ in this specification.

More specifically:

FIG. 1A illustrates an inoculation schedule and dosage of inoculant

FIG. 1B illustrates a comparison of tumor volumes between a control and SAIT-DRP-001.

FIG. 1C illustrates a comparison of tumor weights between a control and SAIT-DRP-001

FIG. 1D illustrates tumor volumes over days of treatment for the treatment groups

FIG. 1E illustrates bodyweight variations of treatment groups.

**** denotes p<0.0001 and, * denotes p<0.1, ns denotes non-significant compared to the indicated respective group. The abbreviation ‘UT’ stands for the word ‘untreated’. Both “UT” and “control” are interchangeably used in this specification.

FIGS. 2A to 2D schematically illustrate the anti-tumor effects of SAIT-DRP-001 alone and along with the gemcitabine combination in KPC tumor-bearing pancreatic xenograft mouse models: Mice bearing KPC orthotopic pancreatic tumors were treated with four different treatment group's control, gemcitabine (Gem), SAIT-DRP-001 and SAIT-DRP-001+Gem. More specifically:

FIG. 2A illustrates a treatment regime of different treatment groups in orthotopic KPC tumor model.

FIG. 2B illustrates tumor volumes.

FIG. 2C illustrates tumor weights.

FIG. 2D illustrates bodyweight variations of treatment groups.

**** denotes p<0.0001 and, * denotes p<0.1, ns denotes non-significant compared to the indicated respective group.

FIG. 3A to 3C schematically illustrate the anti-tumor effects of SAIT-DRP-001 alone and along with the gemcitabine combination in PANC-1 tumor-bearing pancreatic xenograft mouse models. Mice-bearing PANC-1 tumors were treated with four different treatment groups control, gemcitabine, SAIT-DRP-001, and SAIT-DRP-001+gemcitabine.

More specifically:

FIG. 3A illustrates a treatment regime of different treatment groups in PANC-1 tumor model.

FIG. 3B illustrates tumor volumes during the period of experimentation for the treatment groups

FIG. 3C illustrates body weight during the period of experimentation with the treatment groups.

FIGS. 3D to 3E schematically illustrate the results of final day of the experiment, wherein the mice were euthanized, tumors isolated, and the tumor volumes (e.g., see FIG. 3D) and tumor weights (e.g., see FIG. 3E) measured.

**** denotes p<0.0001 and, * denotes p<0.1, ns denotes non-significant compared to the indicated respective group.

FIGS. 4A to 4C schematically illustrate the anti-tumor effects of SAIT-DRP-001 alone, when tested for efficacy as a function of dosage, wherein the mice were euthanized, tumors isolated and the tumor volumes (e.g., see FIG. 4B) and tumor weights (e.g., see FIG. 4C) measured. FIG. 4A illustrates the tumor volumes over the period of experimentation.

**** denotes p<0.0001 and, * denotes p<0.1, ns denotes non-significant compared to the indicated respective group.

### DETAILED DESCRIPTION OF THE INVENTION

One or more specific and alternative embodiments of the present invention will now be described with reference to the attached drawings. It shall be apparent to one skilled in the art, however, that this invention may be practised without such specific details. Some of the details may not be described at length so as not to obscure the invention.

### Certain Terminologies

### Formulated/Formulation

The expression “formulated” as that term is used herein means that the active compounds are mixed with appropriate excipients including surfactant/s and carriers to form a solid mixture or a liquid mixture which is then administered in the subject either orally or parenterally. The expression “formulation” as that term is used herein is taken to mean in this disclosure either the formulated first active compound or the combination of formulated first active compound and the formulated second active compound. In practice, medicinal formulations for administration (i.e., pharmaceutical compositions) may include aqueous solutions, syrups, elixirs, powders, granules, tablets, and capsules which typically contain conventional excipients such as binding agents, fillers, lubricants, disintegrants, wetting agents, suspending agents, emulsifying agents, preservatives, buffer salts, flavoring, coloring, and/or sweetening agents.

### Effective Amount

The compositions or formulations are delivered as effective amounts of active compounds. The term “effective amount” as that term is used herein refers to the amount necessary or sufficient of the active compound to realize a desired biologic effect. If a drug's highest safe dose is known already, the treatment regimen is planned accordingly by adjusting the dose to achieve maximal efficacy possible within limit set by highest safe dose.

### Administration

Suitable administration/treatment protocols for treating cancer or tumor in a subject include, for example, administering to the subject an effective amount of the first active compound or a combination of an effective amount of first active compound and an effective amount of second active compound. If the formulation contains balaglitazone as the sole active compound, then the formulated balaglitazone may be administered in any suitable manner known in the art. If the formulation, for example, is a combination of balaglitazone and gemcitabine, then the formulated balaglitazone and formulated gemcitabine are administered sequentially (at various times) or concurrently (at the same time).

In some embodiments, the formulated active compounds are administered intermittently.

In some embodiments, the formulated active compounds are co-administered. The co-administration can be simultaneous or sequential in either order intravenous (i.e., i.v.) through a continuous infusion. When both formulated active compounds are co-administered sequentially the therapeutic agents are administered in two separate administrations that are separated by a “specific period of time.” The term “specific period of time” as that term is used herein is meant to refer to anywhere from 1 hour to 30 days. For example, one of the agents can be administered within the following time periods: about 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 day(s), and/or about 24, 23, 22, 21, 20, 19, 18, 17, 16, 15, 14, 13, 12, 11, 10, 9, 8, 7, 6, 5, 4, 3, 2 or 1 hour(s).

In some embodiments, simultaneous administration means at the same time or within a brief period of time, usually less than 1 hour. A dosing period as that term is used herein is meant to refer to a period of time, during which each member of the composition has been administered at least once. A dosing period is usually about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 day(s), and, in one embodiment, 6, 7, 8, 9, 10, 11, 12, 13, or 14 days, for example, 7 or 14 days.

In certain embodiments, multiple (for example, 2, 3, 4, 5, 6, 7, 8, 9, 10 or more) doses of formulated first active compound and multiple (for example, 2, 3, 4, 5, 6, 7, 8, 9, 10 or more) doses of formulated second active compound may be administered without crossing known safety limits.

In one embodiment, the dose of formulated compound is calculated as mg of the compound/kg body weight.

In another embodiment, the dose of the active compounds is a flat fixed dose that is fixed irrespective of the weight of the subject.

The two formulated first and second active compounds of the present invention may be administered by the same route of administration or by different routes of administration.

In some embodiments, the formulated first active compound is administered orally or parenterally, i.e. intravenously, intramuscularly, subcutaneously, topically, orally, transdermally, intraperitoneally, intraorbitally, by implantation, by inhalation, intrathecally, intraventricularly, or intranasally. Preferably, the formulated first active compound is administered intravenously.

In some embodiments the formulated second active compound is administered parenterally, i.e. intravenously, intramuscularly, subcutaneously, topically, orally, transdermally, intraperitoneally, intraorbitally, by implantation, by inhalation, intrathecally, intraventricularly, or intranasally. Preferably the formulated second active compound is administered intravenously.

### Treat or Treatment

The terms “treat”, “treating”, “treated” or “treatment”, as those terms are used herein, refer to therapeutic treatment wherein the object is to eliminate or lessen symptoms in the subject in which the drug is administered. Beneficial or desired results include, but are not limited to, elimination of symptoms, alleviation of symptoms, diminishment of extent of condition, stabilized (i.e., not worsening) state of condition, delay or slowing of progression of the condition.

### Cancer

As that term is used herein and unless otherwise defined, “cancer” refers to the growth, division, or proliferation of abnormal cells in the body.

### Cancer or Tumors or Carcinoma

These three terms are used in this disclosure interchangeably.

### Synergy or Synergistic Effect

As used herein, the terms “synergy” or “synergistic effect,” as those terms are used herein, when used in connection with a description of the efficacy of a combination of formulated active compounds, means any measured effect of the combination that is greater than the effect predicted from a sum of the effects of the individual formulated compound.

### SAIT-DRP-001

In the disclosure. SAIT-DRP-001 stands for balaglitazone, a partial agonist of PPAR-γ.

### Chemotherapy Drug

A chemotherapy or chemotherapeutic drug, as that term is used herein is meant to refer to a drug used to treat cancer. Chemotherapeutic drugs work by interfering with the cell cycle, which is the process by which cells reproduce. Chemotherapeutic agents kill cancer cells and prevent them from making more cancer cells. In this disclosure, antimetabolite types of chemotherapeutic drugs are used. An example of an antimetabolite drug is gemcitabine. In this disclosure, the expression, “second active drug” as that term is used herein refers to chemotherapy drug, for example, gemcitabine.

### Subject

As used herein, a “subject” is a mammal. Mammals within the scope of the present invention include, for example, farm animals, domestic animals, laboratory animals, etc. Some examples of farm animals include cows, pigs, horses, goats, etc. Some examples of domestic animals include dogs, cats, etc. Some examples of laboratory animals include primates, rats, mice, rabbits, guinea pigs, etc. Preferably, the mammal is selected from the group consisting of mice, monkeys, rats, and rabbits. Though results on studies on humans as mammalian subject are not presented in this disclosure, the scope would also include humans as a “subject.” The focus of the present invention is on an effective treatment for cancer, in particular, pancreatic cancer and renal cancer.

### Pancreatic Cancer

Treatment of pancreatic cancer depends on the stage of the cancer. Although only localized cancer is considered suitable for surgery with curative intent at present, only about 0.20% of cases present with localized disease at diagnosis. Surgery can also be performed for palliation if the malignancy is invading or compressing the duodenum or colon. In such cases, bypass surgery might overcome the obstruction and improve quality of life but is not intended as a cure. For a disease that is deemed not suitable for resection, palliative chemotherapy may be used to improve the quality of life and gain a modest survival benefit for the patient. There is a need for improved methods for treating pancreatic cancer, in particular, locally advanced and metastatic pancreatic cancer. Metastasis is the leading cause of mortality in cancer patients. However, there are no effective therapeutic methods to target the development and progression of metastases in pancreatic cancer.

### Kidney Cancer or Renal Cancer

If detected early, kidney cancer is curable. However, symptoms may not appear until the tumor has grown to a large size or metastasized to other organs, at which point treatment is palliative. The majority of kidney cancers are renal cell carcinomas (which accounts for over 90% of malignant kidney tumors), also known as renal adenocarcinomas or clear cell carcinomas. Treatment of kidney cancer in individuals whose tumor is confined to the kidney may involve surgical removal of the kidney (i.e., nephrectomy) and surrounding tissue. Therefore, there is a need for improved therapeutic methods for treating kidney cancer as well.

### Chemotherapy Drugs

Chemotherapy drugs are compounds meant to kill cancer cells. Various chemotherapy drugs use different methods to disrupt the cell cycle and fight cancer. The types of chemotherapy drugs include: a) antimetabolites; b) alkylating agents including nitrosoureas; c) topoisomerase inhibitors; d) mitotic inhibitors (plant alkaloids); e) antitumor antibiotics (including anthracyclines); and f) other chemotherapy drugs which fall outside the above types. The inventors have preferred antimetabolite compounds for use as chemotherapy drugs (second active compound used in this invention). Antimetabolites prevent cancer cells from making genetic material which is needed to create new cells. A list of such antimetabolites is mentioned below. The second active compound used in this invention is selected from a list of antimetabolite drugs consisting of gemcitabine, 5-fluorouracil, 6-mercaptopurine, azacitidine, capecitabine, cladribine, clofarabine, cytarabine, decitabine, floxuridine, fludarabine, hydroxyurea, methotrexate, nelarabine, pemetrexed, pentostatin, pralatrexate, thioguanine, trifluridine/tipiracil combination and a combination thereof. The preferred second active compound of the present invention is gemcitabine.

### EXEMPLARY EMBODIMENTS OF THE PRESENT INVENTION

The present invention discloses a formulation and a method of treating a carcinoma selected from a group consisting of renal carcinoma and pancreatic carcinoma in a subject in need thereof.

In one embodiment, the method comprises administering to the subject a formulation comprising a formulated first active compound wherein, the first active compound is a thiazolidinedione-based partial agonist of PPAR-γ.

In another embodiment, the method comprises administering to the subject a formulation comprising a synergistic combination of the formulated first active compound and a formulated second active compound wherein, the second active compound is a chemotherapy drug.

The formulation is a solid or a liquid in form.

In one embodiment, if the formulation contains the first active compound and not the second active compound, wherein the formulated first active compound is administered orally or parenterally.

In another embodiment, the formulated second active compound is administered parenterally.

In one embodiment, the parenteral administration comprises a subcutaneous injection, an intraperitoneal injection, an intravenous injection, an intravenous infusion or an intramuscular injection.

In one embodiment, when the formulation is a synergistic combination of the formulated first active compound and the formulated second active compound, the formulated first active compound and the formulated second active compound of the composition are administered simultaneously or in another embodiment administered sequentially or in yet another embodiment administered in alternating series or in still yet another embodiment administered in temporal proximity.

The formulated first active compound or the formulated second active compound further comprises one or more pharmaceutically acceptable carriers, excipients, or diluents.

In one embodiment, the subject in need to treatment or administration of drug is a mammal.

In another embodiment, mammalian subject is selected from a group consisting of mice, monkeys, rats, rabbits and humans.

Methodology of Testing the Activity of Compositions Used in this Invention

In this section, there is presented a description of the method of in vivo testing of activity of the compositions used in the examples and comparative examples of the present invention.

Animals: The animals used in this study, specifically six- to eight-week-old SCID and C57BL/6 mice, were sourced from our in-house breeding program and housed in our institutional animal facilities. It is crucial to note that all animal experiments were conducted with utmost care and in strict accordance with the guidelines set by the Association for Assessment and Accreditation of Laboratory Animal Care (AAALAC). These experiments were also carried out under protocols that were approved by the Mayo Clinic Institutional Animal Care and Use Committee (IACUC), further ensuring the ethical conduct of our research.

Cell lines: Pancreatic cancer cell lines PANC1 and KPC and kidney cancer cell line 786-O were procured from the American Type Culture Collection (ATCC®, Bethesda, MD, USA). PANC-1, KPC, and 786-O cells were maintained in Dulbecco's Modified Eagle Medium (Gibco DMEM) supplemented with 10% fetal bovine serum (Gibco FBS), 1% Pen Strep (Gibco), and 0.01% Plasmocin (Invivogen, USA).

Formulated first active compound: 5 mg of SAIT-DRP-001 was dissolved in 20 μL of DMSO. Ensure the compound is completely dissolved. If not, warm it to achieve a clear solution, then dissolve it in 1 mL of a PEG mixture. (PEG-mixture is made by mixing PEG-400: Tween80: Ethanol taken in a ratio of 75:8:17).

Formulated second active compound: 2 mg of gemcitabine (MedChem Express, cat. #HY-B0003) was dissolved in 5 mL of 0.9% saline.

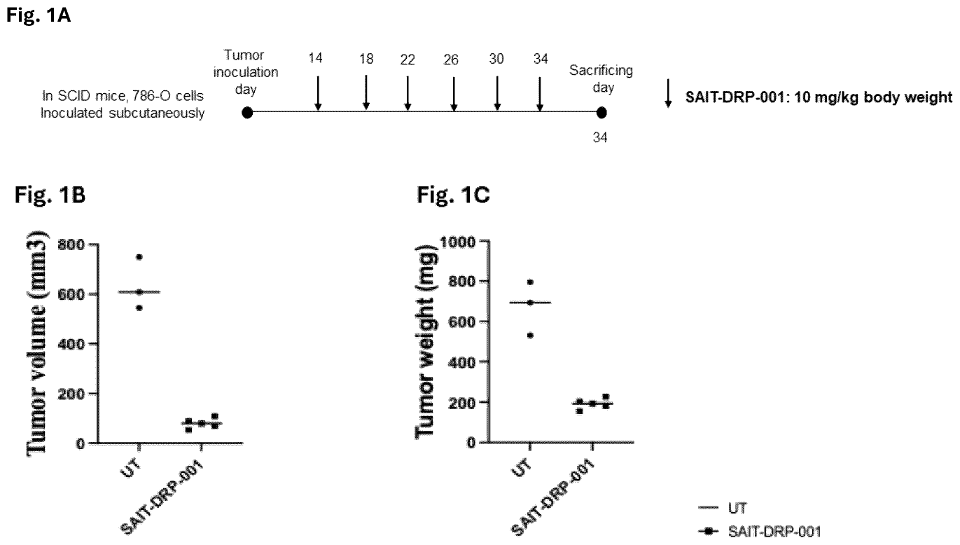

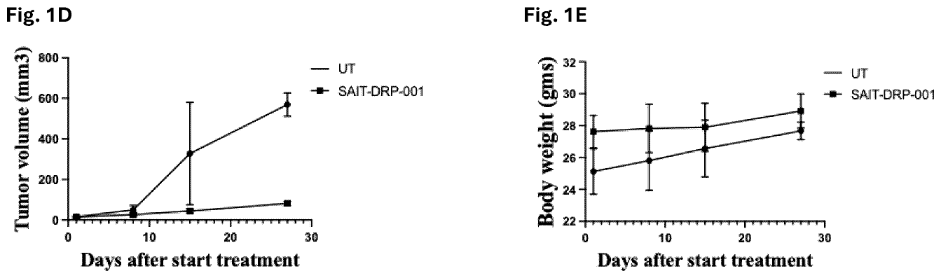

In vivo tumor regression experiment in subcutaneous 786-O tumors: The in vivo tumor regression efficacy of SAIT-DRP-001 was analyzed in xenograft subcutaneous 786-O tumors developed in SCID mice (n=5 per treatment group). For subcutaneous tumor cell implantation, SCID mice were anesthetized with intraperitoneal (i.p.) administration of ketamine/xylazine. The fur on the right flank of the mice was shaved, and the shaved area was disinfected with iodine solution and 70% alcohol. Then, 2×10⁶ 786-O cells suspended in 100 μl sterile PBS were injected subcutaneously into the shaved area of each mouse using a 1 ml syringe equipped with a 26-gauge needle. Following injection, mice were transferred to their cage and kept under a heating lamp for recovery. Mice were observed daily, and treatment was started when the average tumor volume reached ˜50 mm³. SAIT-DRP-001 (10 mg/kg), and another group received a placebo. Tumors were measured weekly with callipers, and tumor volumes were calculated using the formula: Volume=0.5×a×b2, where a and b have the longest and shortest diameters, respectively. Tumor growth curves were obtained by plotting tumor volumes against time. Finally, mice were sacrificed, and the tumors were recorded and the tumor weights and harvested the tumors for immunohistochemistry. The results of this experiment are illustrated in FIGS. 1A to 1E.

### In Vivo Tumor Regression Experiment in Orthotopic KPC Tumors:

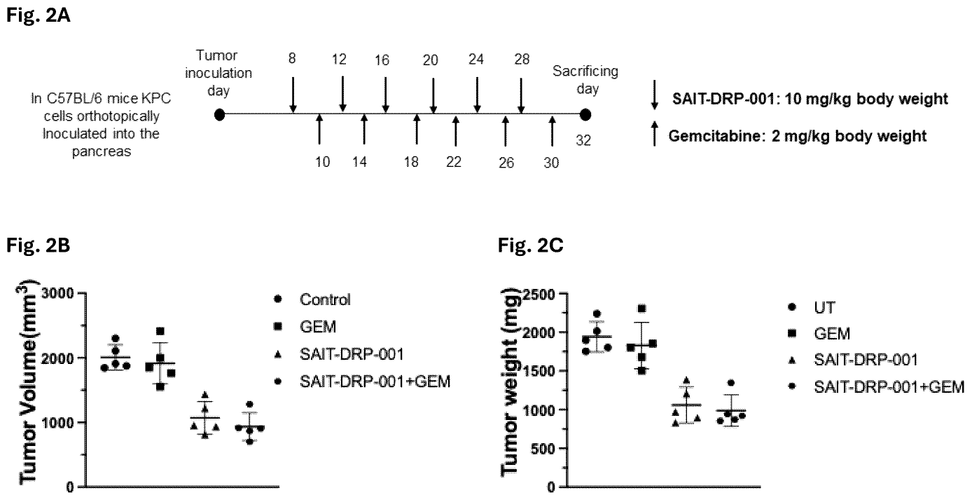

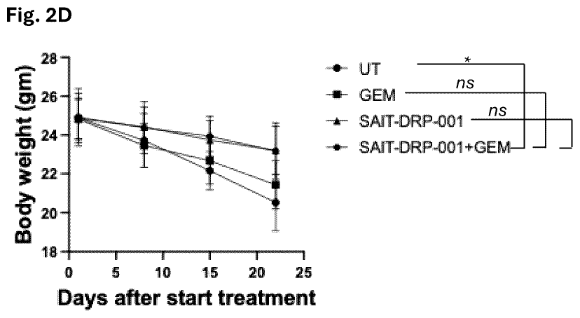

The effectiveness of SAIT-DRP-001 (balaglitazone) in shrinking tumors was tested in KPC tumors grown in C57BL/6 mice (n=5 per treatment group). To implant the tumor cells, C57BL/6 mice were anesthetized using intraperitoneal (i.e., i.p.) administration of ketamine and xylazine. Approximately 0.05×10⁵ luciferase transfected KPC cells suspended in 50 μL of 50% Matrigel were injected into the head of the pancreas of the mouse. After confirming the presence of tumors using IVIS imaging, the mice were divided into four groups and treated with SAIT-DRP-001 (10 mg/kg) and gemcitabine (2 mg/kg), with the combination being administered intraperitoneally twice a week for three weeks. Another group received a placebo. Finally, the mice were euthanized, and the tumors were measured, weighed, and used for immunohistochemistry. The results of this experiment are illustrated in FIGS. 2A to 2D.

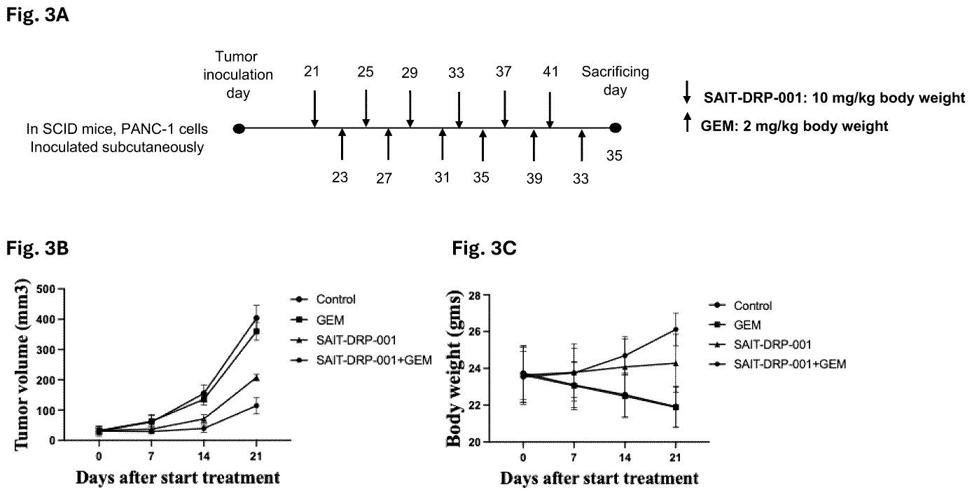

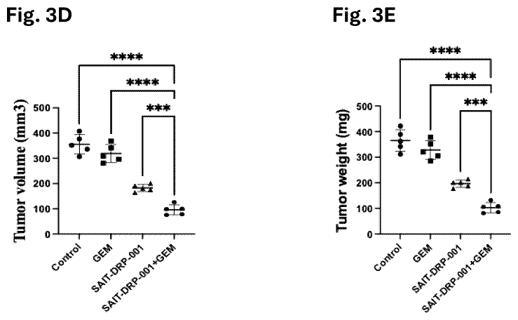

In vivo tumor regression experiment in subcutaneous PANC-1 tumors: The in vivo tumor regression efficacy of SAIT-DRP-001 was analysed in xenograft subcutaneous PANC-1 tumors developed in SCID mice (n=5 per treatment group). For subcutaneous tumor cell implantation, SCID mice were anesthetized with intraperitoneal (i.p.) administration of ketamine/xylazine. The fur on the right flank of the mice was shaved off, and the shaved area was disinfected with iodine solution and 70% alcohol. Then 5×10⁶ PANC-1 cells suspended in 100 μl sterile PBS was injected subcutaneously into the shaved area of each mouse using a 1 ml syringe equipped with a 26-gauge needle. Following injection, mice were transferred to their cage and kept under a heating lamp for recovery. Mice were observed daily, and treatment was started when the average tumor volume reached ˜50 mm³. SAIT-DRP-001 (10 mg/kg), gemcitabine (2 mg/kg), and the combination were administered intraperitoneally twice a week for three weeks. Another group received a placebo. Tumors were measured weekly with calipers, and tumor volumes were calculated using the formula: Volume=0.5×a×b², where a and b have the longest and shortest diameters, respectively. Tumor growth curves were obtained by plotting tumor volumes against time. Finally, mice were sacrificed, the tumor weights recorded, and tumors harvested for immunohistochemistry. The results of this experiment are illustrated in FIGS. 3A to 3E.

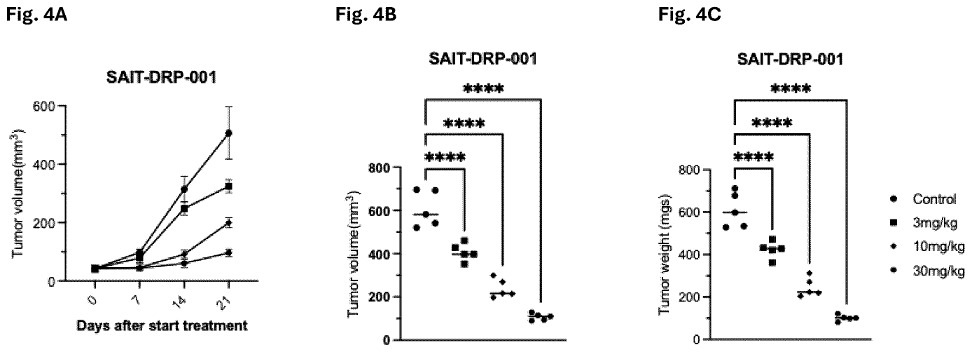

Efficacy of SAIT-DRP-001 in tumor reduction: Mice bearing PANC1 tumors were treated with different doses of SAIT-DRP-001 at 3, 10 and 30 mg/kg body weight. The results are given in FIG. 4B and FIG. 4C where FIG. 4B shows tumor volume and FIG. 4C shows tumor weight. As shown in FIGS. 4A, 4B and 4C, SAIT-DRP-001 demonstrated a dose-dependent antitumor effect in the PANC-1 cell line-induced xenograft model. Significant inhibition of both tumor volume and tumor weight was observed starting from the 3 mg/kg dose. The maximum therapeutic efficacy was achieved at the 30 mg/kg dose, indicating a clear correlation between increasing dosage and tumor growth suppression.

Immunohistochemistry: Tumors were harvested and fixed in neutral buffered 10% formalin at room temperature for 24 hours. Then, they were embedded in paraffin, and 5 μm thick sections were cut for preparing slides. Hematoxylin and eosin (H&E), Ki67 (1:1000) staining were performed in deparaffinized slides as applicable following the manufacturer's instructions (DAB 150; Millipore). Slides were stained with stable diaminobenzidine and counterstained with hematoxylin. Finally, slides were digitized using an Aperio AT2 slide scanner (Leica) and analysed using ImageScope software (Leica). A total of 30 visual fields (10 fields 0.25 mm² each from 3 different tumor sections) were analysed for quantification.

### EXAMPLES AND COMPARATIVE EXAMPLES OF THE PRESENT INVENTION

“Examples of the invention” represent inventive examples of the present invention. Examples and comparative examples of the present invention are listed in the Table. For the examples and comparative examples, the dosage chosen for SAIT-DRP-001 was 10 mg/Kg unless specifically mentioned. The dosage chosen for gemcitabine was 2 mg/Kg. The testing was done as per the procedures given in the previous section. The results of the experiments are illustrated in FIGS. 1A to 1E, 2A-2D, 3A-3E and 4A-4C.

In the first set of experiments (e.g., see the Table, below), balaglitazone is the first active compound and the only active compound present in the formulation. The results, as depicted in FIGS. 1A to 1E, clearly indicate that balaglitazone itself is active against pancreatic cancer in vivo. The data on tumor volume and weight as a function of time reveal that the activity of balaglitazone is significantly different from the control experiment. There is not believed to be a report in the prior art on the activity of balaglitazone in pancreatic cancer by in vivo or in vitro testing.

TABLE
Examples and comparative examples of the present invention

| | Example or | | | | | |
|---|---|---|---|---|---|---|
| | comparative | | Cancer | | First | Second |
| Experiment | example | Subject | cells | | active | active |
| set | reference | Mice | injected | Inoculation | compound | compound |
| 1 | Control | SCID | 786-O | Subcutaneous | None | None |
| | (comparative | | cells | | | |
| | example) | | | | | |
| | SAIT-DRP- | SCID | 786-O | Subcutaneous | SAIT- | None |
| | 001 | | cells | | DRP-001 | |
| | (example) | | | | | |
| 2 | Control | C57BL/6 | KPC | Pancreas | none | None |
| | (comparative | | cells | | | |
| | example) | | | | | |
| | Gem | C57BL/6 | KPC | Pancreas | none | gemcitabine |
| | (Comparative | | cells | | | |
| | example) | | | | | |
| | SAIT-DRP- | C57BL/6 | KPC | Pancreas | SAIT- | None |
| | 001 | | cells | | DRP-001 | |
| | (example) | | | | | |
| | SAIT-DRP- | C57BL/6 | KPC | Pancreas | SAIT- | gemcitabine |
| | 001 + Gem | | cells | | DRP-001 | |
| | (example) | | | | | |
| 3 | Control | SCID | PANC-1 | Subcutaneous | none | None |
| | (comparative | | | | | |
| | example) | | | | | |
| | Gem | SCID | PANC-1 | Subcutaneous | none | gemcitabine |
| | (comparative | | | | | |
| | example) | | | | | |
| | SAIT-DRP- | SCID | PANC-1 | Subcutaneous | SAIT- | None |
| | 001 | | | | DRP-001 | |
| | (example) | | | | | |
| | SAIT-DRP- | SCID | PANC-1 | Subcutaneous | SAIT- | gemcitabine |
| | 001 + Gem | | | | DRP-001 | |
| | (example) | | | | | |
| 4 | Control | SCID | PANC-1 | Subcutaneous | none | None |
| | (comparative | | | | | |
| | example) | | | | | |
| | SAIT-DRP- | SCID | PANC-1 | Subcutaneous | SAIT- | None |
| | 001 (3 | | | | DRP-001 | |
| | mg/Kg) | | | | | |
| | (example) | | | | | |
| | SAIT-DRP- | SCID | PANC-1 | Subcutaneous | SAIT- | None |
| | 001 (10 | | | | DRP-001 | |
| | mg/Kg) | | | | | |
| | (example) | | | | | |
| | SAIT-DRP- | SCID | PANC-1 | Subcutaneous | SAIT- | None |
| | 001 (30 | | | | DRP-001 | |
| | mg/Kg) | | | | | |
| | (example) | | | | | |

In the second set of experiments (e.g., see Table) conducted with a different cancer cell inoculation (KPC cell) in an orthotopic model, where cancer cell loading is aggressive, balaglitazone's activity is quite significant when used as a single active species in the formulation. At the dosage level employed, the activity of gemcitabine is not statistically different from the control experiment. Also, synergistic activity was not statistically significant for the combination of balaglitazone and gemcitabine in KPC cells at the dosage of gemcitabine employed. The nature of cancer cell, cancer cell loading, and the dosage level chosen for gemcitabine are all factors which influence activity of the combination of first and second active compounds.

In the third set of experiments (e.g., see Table) with inoculation of PANC-1 cells, where the cancer cell loading is not aggressive, the activities of both for the first active compound when used alone as well as for the combination of the first active compound and the second active compound are significant. Synergistic activity as shown in FIGS. 3A to 3E, as reflected by the tumor volumes and tumor weights as a function of time, is significant.

Irrespective of the cancer cell loading and the variety of cancer cells employed, balaglitazone seems to exhibit significant anti-cancer activity.

In the fourth set of experiments (see Table) with inoculation of PANC-1 cells, a clear correlation between increasing dosage and tumor growth suppression is seen indicating that balaglitazone itself is significantly specific in activity against cancer cells. FIG. 4A illustrates the high efficacy shown by balaglitazone in activity against cancer cells in a dose-dependent manner.

The overall results indicate that balaglitazone exhibits anti-cancer activity both when used as a single active compound as well as in a combination with gemcitabine. When balaglitazone is used in combination with gemcitabine, synergistic activity is noticed in appropriate cell-specific conditions. The inventiveness of the present invention is highlighted by these surprising results.

Thus, the present invention provides a new potential solution for cancer, particularly pancreatic and renal cancer.

Although various aspects and embodiments of the present invention have been described above, it should be noted that the scope of the invention is by no means limited to the exemplary embodiments described above. The description of the exemplary embodiments of the present invention merely serves to aid in the understanding of the principle underlying the present invention. Thus, the present invention is not to be construed as being limited to the illustrated embodiments. Many changes, modifications, variations and combinations of variations disclosed in the description and drawings thereof could be made to the present invention without departing from the scope of the present invention.

### REFERENCES

1. Gillespie, W., Tyagi, N. and Tyagi, S. C., 2011. Role of PPAR-gamma, a nuclear hormone receptor in neuroprotection. Indian J Biochem Biophys, 48 (2), pp. 73-81. https://pubmed.ncbi.nlm.nih.gov/21682137/2.
2. Celi, F. S. and Shuldiner, A. R., 2002. The role of peroxisome proliferator-activated receptor gamma in diabetes and obesity. Current diabetes reports, 2 (2), pp. 179-185. https://link.springer.com/article/10.1007/s11892-002-0078-2
3. Kapadia, R., Yi, J. H. and Vemuganti, R., 2008. Mechanisms of anti-inflammatory and neuroprotective actions of PPAR-gamma agonists. Frontiers in bioscience: a journal and virtual library, 13, p. 1813. https://doi.org/10.2741% 2F2802
4. Spencer, C. M. and Markham, A., 1997. Troglitazone. Drugs, 54, pp. 89-101. https://link.springer.com/article/10.2165/00003495-199754010-00010
5. Watkins, P. B. and Whitcomb, R. W., 1998. Hepatic dysfunction associated with troglitazone. New England Journal of Medicine, 338 (13), pp. 916-917. https://www.nejm.org/doi/full/10.1056/NEJM199803263381314 #: ˜: text=10.1056/NEJM199803263381314
6. Bhushan, S., Ray, R. S., Prakash, J. and Singh, G. N., 2019. Global versus Indian perspective of pioglitazone-induced adverse drug reactions including bladder cancer: A comparative retrospective pharmacovigilance analysis. Clinical Therapeutics, 41 (11), pp. 2252-2262. https://doi.org/10.1016/j.clinthera.2019.08.018
7. Lebovitz, H. E., Dole, J. F., Patwardhan, R., Rappaport, E. B., Freed, M. I. and Rosiglitazone Clinical Trials Study Group, 2001. Rosiglitazone monotherapy is effective in patients with type 2 diabetes. The Journal of Clinical Endocrinology & Metabolism, 86 (1), pp. 280-288. https://doi.org/10.1210/jcem.86.1.7157
8. Pouwels, K. B. and van Grootheest, K., 2012. The rosiglitazone decision process at FDA and EMA. What should we learn?. International Journal of Risk & Safety in Medicine, 24 (2), pp. 73-80. https://doi.org/10.3233/jrs-2012-0559
9. Sangeeta Ballav, Bini Biswas, Vishal Kumar Sahu, Amit Ranjan and Soumya Basu 2022. PPAR-γ Partial Agonists in Disease-Fate Decision with Special Reference to Cancer. Cells 2022, 11, 3215. https://doi.org/10.3390/cells11203215
10. Kroker A J, Bruning J B. Review of the Structural and Dynamic Mechanisms of PPARγ Partial Agonism. PPAR Res. 2015; 2015:816856. doi: 10.1155/2015/816856.
11. Ballav, S., Biswas, B., Sahu, V. K., Ranjan, A. and Basu, S., 2022. PPAR-γ partial agonists in disease-fate decision with reference special to cancer. Cells, 11 (20), p. 3215. https://doi.org/10.3390% 2Fcells11203215
12. Agrawal, R., Jain, P. and N Dikshit, S., 2012. Balaglitazone: a second generation peroxisome proliferator-activated receptor (PPAR) gamma (γ) agonist. Mini reviews in medicinal chemistry, 12 (2), pp. 87-97. https://doi.org/10.2174/138955712798995048
13. https://en.wikipedia.org/wiki/Thiazolidinedione #: ˜:text=Troglitazone % 20 (Rezulin)%2C%20withdrawn%20due,no%20better%20than%20available%20molecules. Accessed on 22nd July 2024.
14. Henriksen, K., Byrjalsen, I., Qvist, P., Beck-Nielsen, H., Hansen, G., Riis, B. J., Perrild, H., Svendsen, O. L., Gram, J., Karsdal, M. A. and Christiansen, C., 2011. Efficacy and safety of the PPARγ partial agonist balaglitazone compared with pioglitazone and placebo: a phase III, randomized, parallel-group study in patients with type 2 diabetes on stable insulin therapy. Diabetes/metabolism research and reviews, 27 (4), pp. 392-401. https://doi.org/10.1002/dmrr.1187
15. Hernandez-Quiles, M., Broekema, M. F. and Kalkhoven, E., 2021. PPAR-gamma in metabolism, immunity, and cancer: unified and diverse mechanisms of action. Frontiers in endocrinology, 12, p. 624112. https://doi.org/10.3389/fendo.2021.624112
16. Grommes, C., Landreth, G. E. and Heneka, M. T., 2004. Antineoplastic effects of peroxisome proliferatoractivated receptor γ agonists. The lancet oncology, 5 (7), pp. 419-429.
17. Huang, J. W., Shiau, C. W., Yang, Y. T., Kulp, S. K., Chen, K. F., Brueggemeier, R. W., Shapiro, C. L. and Chen, C. S., 2005. Peroxisome proliferator-activated receptor γ-independent ablation of cyclin D1 by thiazolidinediones and their derivatives in breast cancer cells. Molecular pharmacology, 67 (4), pp. 1342-1348. https://doi.org/10.1124/mol.104.007732
18. Lyles, B. E., Akinyeke, T. O., Moss, P. E. and Stewart, L. V., 2009. Thiazolidinediones regulate expression of cell cycle proteins in human prostate cancer cells via PPARγ-dependent and PPARγ-independent pathways. Cell Cycle, 8 (2), pp. 268-277. https://doi.org/10.4161/cc.8.2.7584
19. Botton, T., Puissant, A., Bahadoran, P., Annicotte, J. S., Fajas, L., Ortonne, J. P., Gozzerino, G., Zamoum, T., Tartare-Deckert, S., Bertolotto, C. and Ballotti, R., 2009. In vitro and in vivo anti-melanoma effects of ciglitazone. Journal of Investigative Dermatology, 129 (5), pp. 1208-1218. https://doi.org/10.1038/jid.2008.346
20. Shiau, C. W., Yang, C. C., Kulp, S. K., Chen, K. F., Chen, C. S., Huang, J. W. and Chen, C. S., 2005. Thiazolidenediones mediate apoptosis in prostate cancer cells in part through inhibition of Bcl-xL/Bcl-2 functions independently of PPARγ. Cancer research, 65 (4), pp. 1561-1569. https://doi.org/10.1158/0008-5472.CAN-04-1677
21. Kim, Y., Suh, N., Sporn, M. and Reed, J. C., 2002. An inducible pathway for degradation of FLIP protein sensitizes tumor cells to TRAIL-induced apoptosis. Journal of Biological Chemistry, 277 (25), pp. 22320-22329. https://doi.org/10.1074/jbc.M202458200
22. Qin, C., Burghardt, R., Smith, R., Wormke, M., Stewart, J. and Safe, S., 2003. Peroxisome proliferator-activated receptor γ agonists induce proteasome-dependent degradation of cyclin D1 and estrogen receptor α in MCF-7 breast cancer cells. Cancer research, 63 (5), pp. 958-964. https://aacrjournals.org/cancerres/article/63/5/958/510989/Peroxisome-Proliferator-activated-Receptor
23. Pestereva, E., Kanakasabai, S. and Bright, J. J., 2012. PPARγ agonists regulate the expression of stemness and differentiation genes in brain tumour stem cells. British Journal of Cancer, 106 (10), pp. 1702-1712. https://www.nature.com/articles/bjc2012161
24. Chang, S. N., Lee, J. M., Oh, H., Kim, U., Ryu, B. and Park, J. H., 2018. Troglitazone inhibits the migration and invasion of PC-3 human prostate cancer cells by upregulating E-cadherin and glutathione peroxidase 3. Oncology letters, 16 (4), pp. 5482-5488.

https://doi.org/10.3892/ol.2018.9278
25. Frohlich, E., Machicao, F. and Wahl, R., 2005. Action of thiazolidinediones on differentiation, proliferation and apoptosis of normal and transformed thyrocytes in culture. Endocrine-Related Cancer, 12 (2), pp. 291-303. https://doi.org/10.1677/erc.1.00973
26. Elstner, E., Williamson, E. A., Zang, C., Fritz, J., Heber, D., Fenner, M., Possinger, K. and Koeffler, H. P., 2002. Novel therapeutic approach: ligands for PPARγ and retinoid receptors induce apoptosis in bcl-2-positive human breast cancer cells. Breast cancer research and treatment, 74, pp. 155-165. https://link.springer.com/article/10.1023/A: 1016114026769
27. Fujita, M., Hasegawa, A., Yamamori, M. and Okamura, N., 2017. In vitro and in vivo cytotoxicity of troglitazone in pancreatic cancer. Journal of Experimental & Clinical Cancer Research, 36, pp. 1-9. https://link.springer.com/article/10.1186/s13046-017-0557-6
28. Reddy, R. C., Srirangam, A., Reddy, K., Chen, J., Gangireddy, S., Kalemkerian, G. P., Standiford, T. J. and Keshamouni, V. G., 2008. Chemotherapeutic drugs induce PPAR-γ expression and show sequence-specific synergy with PPAR-γ ligands in inhibition of non-small cell lung cancer. Neoplasia, 10 (6), pp. 597-603. https://doi.org/10.1593/neo.08134
29. Bräutigam, K., Biernath-Wüpping, J., Bauerschlag, D. O., Von Kaisenberg, C. S., Jonat, W., Maass, N., Arnold, N. and Meinhold-Heerlein, I., 2011. Combined treatment with TRAIL and PPARγ ligands overcomes chemoresistance of ovarian cancer cell lines. Journal of Cancer Research and Clinical Oncology, 137, 875-886. pp.

https://link.springer.com/article/10.1007/s00432-010-0952-2
30. Park, B. H., Lee, S. B., Stolz, D. B., Lee, Y. J. and Lee, B. C., 2011. Synergistic interactions between heregulin and peroxisome proliferator-activated receptor-γ (PPARγ) agonist in breast cancer cells. Journal of Biological Chemistry, 286 (22), pp. 20087-20099. https://doi.org/10.1074/jbc.M110.191718
31. Liu, Y., Zhu, Z. A., Zhang, S. N., Mou, J., Liu, L., Cui, T. and Pei, D. S., 2013. Combinational effect of PPARγ agonist and RXR agonist on the growth of SGC7901 gastric carcinoma cells in vitro. Tumor Biology, 34, pp. 2409-2418. https://link.springer.com/article/10.1007/s13277-013-0791-2
32. Hamaguchi, N., Hamada, H., Miyoshi, S., Irifune, K., Ito, R., Miyazaki, T. and Higaki, J., 2010. In vitro and in vivo therapeutic efficacy of the PPAR-γ agonist troglitazone in combination with cisplatin against malignant pleural mesothelioma cell growth. Cancer science, 101 (9), pp. 1955-1964. https://doi.org/10.1111/j.1349-7006.2010.01632.x
33. Mueller, E., Smith, M., Sarraf, P., Kroll, T., Aiyer, A., Kaufman, D. S., Oh, W., Demetri, G., Figg, W. D., Zhou, X. P. and Eng, C., 2000. Effects of ligand activation of peroxisome proliferator-activated receptor γ in human prostate cancer. Proceedings of the National Academy of Sciences, 97 (20), pp. 10990-10995. https://doi.org/10.1073/pnas.180329197
34. Kohno, H., Yoshitani, S. I., Takashima, S., Okumura, A., Hosokawa, M., Yamaguchi, N. and Tanaka, T., 2001. Troglitazone, a Ligand for Peroxisome Proliferator-activated Receptor Y Inhibits Chemically-induced Aberrant Crypt Foci in Rats. Japanese Journal of Cancer Research, 92 (4), pp. 396-403. https://doi.org/10.1111/j.1349-7006.2001.tb01108.x
35. Yoshida, K., Hirose, Y., Tanaka, T., Yamada, Y., Kuno, T., Kohno, H., Katayama, M., Qiao, Z., Sakata, K., Sugie, S. and Shibata, T., 2003. Inhibitory effects of troglitazone, a peroxisome proliferator-activated receptor γ ligand, in rat tongue carcinogenesis initiated with 4-nitroquinoline 1-oxide. Cancer science, 94 (4), pp. 365-371. https://doi.org/10.1111/j.1349-7006.2003.tb01448.x
36. Wan, Z., Shi, W., Shao, B., Shi, J., Shen, A., Ma, Y., Chen, J. and Lan, Q., 2011. Peroxisome proliferator-activated receptor γ agonist pioglitazone inhibits β-catenin-mediated glioma cell growth and invasion. Molecular and cellular biochemistry, 349, pp. 1-10. https://link.springer.com/article/10.1007/s11010-010-0637-9
37. Ninomiya, I., Yamazaki, K., Oyama, K., Hayashi, H., Tajima, H., Kitagawa, H., Fushida, S., Fujimura, T. and Ohta, T., 2014. Pioglitazone inhibits the proliferation and metastasis of human pancreatic cancer cells. Oncology Letters, 8 (6), pp. 2709-2714. https://www.spandidos-publications.com/ol/8/6/2709
38. Yang, Y., Zhao, L. H., Huang, B., Wang, R. Y., Yuan, S. X., Tao, Q. F., Xu, Y., Sun, H. Y., Lin, C. and Zhou, W. P., 2015. Pioglitazone, a PPARγ agonist, inhibits growth and invasion of human hepatocellular carcinoma via blockade of the rage signaling. Molecular carcinogenesis, 54 (12), pp. 1584-1595. https://doi.org/10.1002/mc.22231
39. Srivastava, N., Kollipara, R. K., Singh, D. K., Sudderth, J., Hu, Z., Nguyen, H., Wang, S., Humphries, C. G., Carstens, R., Huffman, K. E. and DeBerardinis, R. J., 2014. Inhibition of cancer cell proliferation by PPARγ is mediated by a metabolic switch that increases reactive oxygen species levels. Cell metabolism, 20 (4), pp. 650-661. https://doi.org/10.1016/j.cmet.2014.08.003
40. Takano, S., Kubota, T., Nishibori, H., Hasegawa, H., Ishii, Y., Nitori, N., Ochiai, H., Okabayashi, K., Kitagawa, Y., Watanabe, M. and Kitajima, M., 2008. Pioglitazone, a ligand for peroxisome proliferator-activated receptor-γ acts as an inhibitor of colon cancer liver metastasis. Anticancer Research, 28 (6A), pp. 3593-3599. https://ar.iiarjournals.org/content/28/6A/3593.short
41. Cramer, C. K., Alphonse-Sullivan, N., Isom, S., Metheny-Barlow, L. J., Cummings, T. L., Page, B. R., Brown, D. R., Blackstock, A. W., Peiffer, A. M., Strowd, R. E. and Rapp, S., 2019. Safety of pioglitazone during and after radiation therapy in patients with brain tumors: a phase I clinical trial. Journal of cancer research and clinical oncology, 145, pp. 337-344. https://link.springer.com/article/10.1007/s00432-018-2791-5
42. Reichle, A., Bross, K., Vogt, T., Bataille, F., Wild, P., Berand, A., Krause, S. W. and Andreesen, R., 2004. Pioglitazone and rofecoxib combined with angiostatically scheduled trofosfamide in the treatment of far-advanced melanoma and soft tissue sarcoma. Cancer: Interdisciplinary International Journal of the American Cancer Society, 101 (10), pp. 2247-2256. https://doi.org/10.1002/cncr.20574
43. Vogt, T., Hafner, C., Bross, K., Bataille, F., Jauch, K. W., Berand, A., Landthaler, M., Andreesen, R. and Reichle, A., 2003. Antiangiogenetic therapy with pioglitazone, rofecoxib, and metronomic trofosfamide in patients with advanced malignant vascular tumors. Cancer: Interdisciplinary International Journal of the American Cancer Society, 98 (10), pp. 2251-2256. https://doi.org/10.1002/cncr.11775
44. Cao, L. Q., Wang, X. L., Wang, Q., Xue, P., Jiao, X. Y., Peng, H. P., Lu, H. W., Zheng, Q., Chen, X. L., Huang, X. H. and Fu, X. H., 2009. Rosiglitazone sensitizes hepatocellular carcinoma cell lines to 5-fluorouracil antitumor activity through activation of the PPARγ signaling pathway. Acta Pharmacologica Sinica, 30 (9), pp. 1316-1322. https://www.nature.com/articles/aps2009119
45. Hsu, M. C., Huang, C. C., Chang, H. C., Hu, T. H. and Hung, W. C., 2008. Overexpression of Jabl in hepatocellular carcinoma and its inhibition by peroxisome proliferator-activated receptory ligands in vitro and in vivo. Clinical cancer research, 14 (13), pp. 4045-4052. https://doi.org/10.1158/1078-0432.CCR-07-5040
46. Kim, Y. H., Jung, E. M., Lee, T. J., Kim, S. H., Choi, Y. H., Park, J. W., Park, J. W., Choi, K. S. and Kwon, T. K., 2008. Rosiglitazone promotes tumor necrosis factor-related apoptosis-inducing ligand-induced apoptosis by reactive oxygen species-mediated up-regulation of death receptor 5 and down-regulation of c-FLIP. Free Radical Biology and Medicine, 44 (6), pp. 1055-1068. https://doi.org/10.1016/j.freeradbiomed.2007.12.001
47. Li, Y., Meng, Y., Li, H., Li, J., Fu, J., Liu, Y. and Chen, X., 2006. Growth inhibition and differentiation induced by peroxisome proliferator activated receptor gamma ligand rosiglitazone in human melanoma cell line A375. Medical Oncology, 23, pp. 393-402. https://link.springer.com/article/10.1385/MO: 23:3:393
48. Kumar, A. P., Quake, A. L., Chang, M. K. X., Zhou, T., Lim, K. S. Y., Singh, R., Hewitt, R. E., Salto-Tellez, M., Pervaiz, S. and Clément, M. V., 2009. Repression of NHE1 expression by PPARγ activation is a potential new approach for specific inhibition of the growth of tumor cells in vitro and in vivo. Cancer research, 69 (22), pp. 8636-8644. https://doi.org/10.1158/0008-5472.CAN-09-0219
49. Pignatelli, M., Cocca, C., Santos, A. and Perez-Castillo, A., 2003. Enhancement of BRCA1 gene expression by the peroxisome proliferator-activated receptor γ in the MCF-7 breast cancer cell line. Oncogene, 22 (35), pp. 5446-5450. https://www.nature.com/articles/1206824
50. Sun, W. H., Chen, G. S., Ou, X. L., Yang, Y., Luo, C., Zhang, Y., Shao, Y., Xu, H. C., Xiao, B., Xue, Y. P. and Zhou, S. M., 2009. Inhibition of COX-2 and activation of peroxisome proliferator-activated receptor γ synergistically inhibits proliferation and induces apoptosis of human pancreatic carcinoma cells. Cancer Letters, 275 (2), pp. 247-255. https://doi.org/10.1016/j.canlet.2008.10.023
51. Bunt, S. K., Mohr, A. M., Bailey, J. M., Grandgenett, P. M. and Hollingsworth, M. A., 2013. Rosiglitazone and Gemcitabine in combination reduces immune suppression and modulates T cell populations in pancreatic cancer. Cancer immunology, immunotherapy, 62, pp. 225-236. https://link.springer.com/article/10.1007/s00262-012-1324-3
52. Hazra, S., Batra, R. K., Tai, H. H., Sharma, S., Cui, X. and Dubinett, S. M., 2007. Pioglitazone and rosiglitazone decrease prostaglandin E2 in non-small-cell lung cancer cells by up-regulating 15-hydroxyprostaglandin dehydrogenase. Molecular Pharmacology, 71 (6), pp. 1715-1720. https://doi.org/10.1124/mol.106.033357
53. Han, S., Zheng, Y. and Roman, J., 2007. Rosiglitazone, an Agonist of PPARγ, Inhibits Non-Small Cell Carcinoma Cell Proliferation In Part through Activation of Tumor Sclerosis Complex-2. PPAR research, 2007 (1), p. 029632. https://doi.org/10.1155/2007/29632
54. Cerquetti, L., Sampaoli, C., Amendola, D., Bucci, B., Masuelli, L., Marchese, R., Misiti, S., De Venanzi, A., Poggi, M., Toscano, V. and Stigliano, A., 2011. Rosiglitazone induces autophagy in H295R and cell cycle deregulation in SW13 adrenocortical cancer cells. Experimental cell research, 317 (10), pp. 1397-1410. https://doi.org/10.1016/j.yexcr.2011.02.014
55. Qin, L., Gong, C., Chen, A. M., Guo, F. J., Xu, F., Ren, Y. and Liao, H., 2014. Peroxisome proliferator-activated receptor γ agonist rosiglitazone inhibits migration and invasion of prostate cancer cells through inhibition of the CXCR4/CXCL12 axis. Molecular medicine reports, 10 (2), pp. 695-700. https://doi.org/10.3892/mmr.2014.2232
56. Miao, R., Xu, T. A. O., Liu, L., Wang, M., Jiang, Y., Li, J. and Guo, R., 2011. Rosiglitazone and retinoic acid inhibit proliferation and induce apoptosis in the HCT-15 human colorectal cancer cell line. ExpErimEntal and thErapEutic mEdicinE, 2 (3), pp. 413-417. https://doi.org/10.3892/etm.2011.227
57. Smith, A. G., Beaumont, K. A., Smit, D. J., Thurber, A. E., Cook, A. L., Boyle, G. M., Parsons, P. G., Sturm, R. A. and Muscat, G. E., 2009. PPARγ agonists attenuate proliferation and modulate Wnt/β-catenin signalling in melanoma cells. The international journal of biochemistry & cell biology, 41 (4), pp. 844-852. https://doi.org/10.1016/j.biocel.2008.08.037
58. Rui, M., Huang, Z., Liu, Y., Wang, Z., Liu, R., Fu, J. and Huang, H., 2014. Rosiglitazone suppresses angiogenesis in multiple myeloma via downregulation of hypoxia-inducible factor-la and insulin-like growth factor-1 mRNA expression. Molecular Medicine Reports, 10 (4), pp. 2137-2143. https://doi.org/10.3892/mmr.2014.2407
59. Chi T, Wang M, Wang X, Yang K, Xie F, Liao Z, Wei P. PPAR-γ Modulators as Current and Potential Cancer Treatments. Front Oncol. 2021 Sep. 23; 11:737776. doi:

10.3389/fonc.2021.737776. PMID: 34631571; PMCID: PMC8495261.
60. Yousefi, B., Azimi, A., Majidinia, M., Shafiei-Irannejad, V., Badalzadeh, R., Baradaran, B., Zarghami, N. and Samadi, N., 2017. Balaglitazone reverses P-glycoprotein-mediated multidrug resistance via upregulation of PTEN in a PPARγ-dependent manner in leukemia cells. Tumor Biology, 39 (10), p. 1010428317716501. https://doi.org/10.1177/1010428317716501
61. WO2018132899A1-Compound, composition, and methods for treating, preventing, reducing, or delaying onset of osteosarcoma lung metastasis in a subject. https://patents.google.com/patent/WO2018132899A1/en?oq-WO2018132899A1
62. Khatik, Gopal & Datusalia, Ashok & Ahsan, Waquar & Kaur, Paranjeet & Vyas, Manish & Mittal, Amit & Nayak, Surendra. (2017). A Retrospect Study on Thiazole Derivatives as the Potential Antidiabetic Agents in Drug Discovery & Developments. Current drug discovery technologies. 14. 10.2174/1570163814666170915134018.
63. KW, Zhang X, Imchen T, Baek S J. A peroxisome proliferator-activated receptor ligand MCC-555 imparts anti-proliferative response in pancreatic cancer cells by PPAR gamma-independent up-regulation of KLF4. Toxicol Appl Pharmacol. 2012 Sep. 1; 263 (2): 225-32. doi: 10.1016/j.taap.2012.06.014. Epub 2012 Jun. 30. PMID: 22750490; PMCID: PMC3443873.
64. Yamaguchi, K. (2006). A novel peroxisome proliferator-activated receptor ligand, MCC-555, induces apoptosis via posttranscriptional regulation of NAG-1 in colorectal cancer cells. Molecular Cancer Therapeutics, 5 (5), 1352-1361. doi: 10.1158/1535-7163.mct-05-0528
64. Bryant C, Rask G, Waller A P, Webb A, Galdino-Pitta M R, Amato A A, Cianciolo R, Govindarajan R, Becknell B, Kerlin B A, Neves F A R, Fornoni A, Agrawal S. Selective modulator of nuclear receptor PPARγ with reduced adipogenic potential ameliorates experimental nephrotic syndrome. iScience. 2022 Feb. 28; 25 (4): 104001. doi: 10.1016/j.isci.2022.104001.
65. Ferreira, Anna & Coelho, Michella & Amato, Angélica & Neves, Francisco & Rodrigues, Izabel & Royer, Carine. (2018). Effect of PPAR Partial Agonist, GQ-16, on Viability of Breast Cancer Cells in Culture. The FASEB Journal. 31. 10.1096/fasebj.31.1_supplement.876.5.
67. Smallridge R C, Copland J A, Brose M S, Wadsworth J T, Houvras Y, Menefee M E, Bible K C, Shah M H, Gramza A W, Klopper J P, Marlow L A, Heckman M G, Von Roemeling R. Efatutazone, an oral PPAR-γ agonist, in combination with paclitaxel in anaplastic thyroid cancer: results of a multicenter phase 1 trial. J Clin Endocrinol Metab. 2013 June; 98 (6): 2392-400. doi: 10.1210/jc.2013-1106. Epub 2013 Apr. 15. PMID: 23589525; PMCID: PMC3667260.
68. https://sci-hub.se/https://doi.org/10.1517/13543784.2015.971154 Accessed on Jul. 18, 2024.
69. Sangeeta Ballav, Bini Biswas, Vishal Kumar Sahu, Amit Ranjan and Soumya Basu (2022). PPAR-γ Partial Agonists in Disease-Fate Decision with Special Reference to Cancer, Cells 2022, 11 (20), 3215; https://doi.org/10.3390/cells11203215.
70. Lee, L. D., Mafura, B., Lauscher, J. C., Seeliger, H., Kreis, M. E., & Gröne, J. (2014). Antiproliferative and apoptotic effects of telmisartan in human colon cancer cells. Oncology Letters, 8, 2681-2686. https://doi.org/10.3892/ol.2014.2592.
71. Kiyoaki funao, Masahide Matsuyama, yutaka kawahito, hajime sano, jamel chargui, jean-louis touraine, tatsuya nakatani and rikio Yoshimura (2008). Telmisartan is a potent target for prevention and treatment in human prostate cancer. ONCOLOGY REPORTS 20:295-300, 2008. DOI: 10.3892/or_00000006.
72. https://ijmsweb.com/an-epidemiological-review-of-pancreatic-cancer-with-special-reference-to-india/
73. https://www.cancer.org/cancer/types/pancreatic-cancer/about/key-statistics.html
74. https://main.icmr.nic.in/sites/default/files/guidelines/consensu_3.pdf
75. Diana, P., Klatte, T., Amparore, D., Bertolo, R., Carbonara, U., Erdem, S., Ingels, A., Kara, O., Marandino, L., Marchioni, M. and Muselaers, S., 2023. Screening programs for renal cell carcinoma: a systematic review by the EAU young academic urologists renal cancer working group. World journal of urology, 41 (4), pp. 929-940.

https://link.springer.com/article/10.1007/s00345-022-03993-6
76. Ferro, M., Musi, G., Marchioni, M., Maggi, M., Veccia, A., Del Giudice, F., Barone, B., Crocetto, F., Lasorsa, F., Antonelli, A. and Schips, L., 2023. Radiogenomics in renal cancer management-current evidence and future prospects. International journal of molecular sciences, 24 (5), p. 4615. https://doi.org/10.3390/ijms24054615
77. https://academic.oup.com/ndt/article-abstract/39/6/920/7606319. Accessed on Jul. 22, 2024.

What is claimed is:

1. A method of treating a carcinoma in a subject in need thereof, wherein the carcinoma includes renal carcinoma or pancreatic carcinoma, comprising the step of:
administering to the subject a formulation;
wherein the formulation comprises:
a formulated first active compound;
wherein the first active compound is a thiazolidinedione-based partial agonist of PPAR-γ; and
optionally, a synergistic combination of the formulated first active compound and a formulated second active compound;
wherein the second active compound is a chemotherapy drug.

2. The method according to claim 1, wherein the formulation is a solid or a liquid in form.

3. The method according to claim 1, wherein the formulated first active compound is administered orally or parenterally.

4. The method according to claim 1, wherein the formulated second active compound is administered parenterally.

5. The method according to claim 4, wherein the parenteral administration comprises a subcutaneous injection, an intraperitoneal injection, an intravenous injection, an intravenous infusion or an intramuscular injection.

6. The method according to claim 1, wherein, when the formulation is a synergistic combination of the formulated first active compound and the formulated second active compound, the formulated first active compound and the formulated second active compound of the composition are administered simultaneously, are administered sequentially, are administered in alternating series or are administered in temporal proximity.

7. The method according to claim 1, wherein the first active compound is a molecule selected from the group consisting of balaglitazone, efatutazone, GQ-16, netoglitazone, and combinations thereof.

8. The method according to claim 1, wherein the second active compound is a molecule selected from the group consisting of gemcitabine, 5-fluorouracil, 6-mercaptopurine, azacitidine, capecitabine, cladribine, clofarabine, cytarabine, decitabine, floxuridine, fludarabine, hydroxyurea, methotrexate, nelarabine, pemetrexed, pentostatin, pralatrexate, thioguanine, trifluridine, tipiracil, and combinations thereof.

9. The method according to claim 1, wherein the formulated first active compound or formulated second active compound further comprises one or more pharmaceutically acceptable carriers, excipients, or diluents.

10. The method according to claim 1, wherein the subject is a mammal.

11. The method according to claim 10, wherein the mammal is selected from the group consisting of mice, monkeys, rats, rabbits, and combinations thereof.

12. A formulation for treating a carcinoma in a subject in need thereof, wherein the carcinoma includes renal carcinoma or pancreatic carcinoma, comprising:
a formulated first active compound;
wherein the first active compound is a thiazolidinedione-based partial agonist of PPAR-γ; and
optionally, a synergistic combination of the formulated first active compound and a formulated second active compound;
wherein the second active compound is a chemotherapy drug.

13. The formulation according to claim 12, wherein the formulation is a solid or a liquid in form.

14. The formulation according to claim 12, wherein the formulated first active compound is administered orally or parenterally.

15. The formulation according to claim 12, wherein the formulated second active compound is administered parenterally.

16. The formulation according to claim 15, wherein the parenteral administration comprises a subcutaneous injection, an intraperitoneal injection, an intravenous injection, an intravenous infusion or an intramuscular injection.

17. The formulation according to claim 12, wherein, when the formulation is a synergistic combination of the formulated first active compound and the formulated second active compound, the formulated first active compound and the formulated second active compound of the composition are administered simultaneously, are administered sequentially, are administered in alternating series or are administered in temporal proximity.

18. The formulation according to claim 12, wherein the first active compound is a molecule selected from the group consisting of balaglitazone, efatutazone, GQ-16, netoglitazone, and combinations thereof.

19. The formulation according to claim 12, wherein the second active compound is a molecule selected from the group consisting of gemcitabine, 5-fluorouracil, 6-mercaptopurine, azacitidine, capecitabine, cladribine, clofarabine, cytarabine, decitabine, floxuridine, fludarabine, hydroxyurea, methotrexate, nelarabine, pemetrexed, pentostatin, pralatrexate, thioguanine, trifluridine, tipiracil, and combinations thereof.

20. The formulation according to claim 12, wherein the formulated first active compound or formulated second active compound further comprises one or more pharmaceutically acceptable carriers, excipients, or diluents.

21. The formulation according to claim 12, wherein the subject is a mammal.

22. The formulation according to claim 21, wherein the mammal is selected from the group consisting of mice, monkeys, rats, rabbits, and combinations thereof.
